(19) United States
(12) Patent Application Publication
(10) Pub. No.: US 2025/0161400 A1
(43) Pub. Date: May 22, 2025

(54) BIOAVAILABLE COMPOSITIONS INCLUDING NANOPARTICULATED INGREDIENTS FOR REDUCING OXIDATIVE STRESS

(71) Applicant: BIOTECH NUTRITIONAL SCIENCES, LLC, Salt Lake City, UT (US)

(72) Inventor: Bert Wonnacott, Salt Lake CIty, UT (US)

(73) Assignee: BIOTECH NUTRITIONAL SCIENCES, LLC, Salt Lake City, UT (US)

(21) Appl. No.: 18/962,522

(22) Filed: Nov. 27, 2024

Related U.S. Application Data
(63) Continuation-in-part of application No. 18/656,465, filed on May 6, 2024.
(60) Provisional application No. 63/500,392, filed on May 5, 2023.

(51) Int. Cl. A61K 38/06; A61K 9/14; A61K 31/12; A61K 31/198; A61K 38/39

(52) U.S. Cl. CPC A61K 38/063; A61K 9/14; A61K 31/12; A61K 31/198; A61K 38/39

(57) ABSTRACT

Bioavailable compositions comprising nanoparticulated ingredients in aqueous solution, methods of making the same, and methods of administering the same. A method includes providing an effective amount of a composition to an animal to reduce oxidative stress in the animal. The composition includes a water solvent and a nanoparticulated ingredient, wherein the nanoparticulated ingredient comprises one or more of nanoparticulated glutathione, nanoparticulated l-cysteine, nanoparticulated curcumin, or nanoparticulated collagen.

### CROSS-REFERENCE TO RELATED APPLICATIONS

This application is a continuation-in-part of U.S. patent application Ser. No. 18/656,465, filed May 6, 2024, entitled “BIOAVAILABLE COMPOSITIONS INCLUDING NANOPARTICULATED INGREDIENTS FOR REDUCING OXIDATIVE STRESS,” which claims the benefit of U.S. Provisional Patent Application No. 63/500,392, filed May 5, 2023. The aforementioned patent applications are incorporated herein by reference in their entireties, including but not limited to those portions that specifically appear hereinafter, the incorporation by reference being made with the following exception: In the event that any portion of the above-referenced patent applications are inconsistent with this application, this application supersedes the above-referenced patent applications.

### TECHNICAL FIELD

The disclosure relates generally to compositions for reducing oxidative stress and specifically to composition comprising nanoscale ingredients.

### BACKGROUND

Oxidative stress occurs when there is an imbalance between free radicals and antioxidants in the body. Free radicals are unstable molecules that can damage cells and contribute to the development of various health problems. Antioxidants are compounds that neutralize free radicals and help protect cells from damage. Oxidative stress can be relieved through supplementation of antioxidants, but traditional supplements fail to deliver antioxidants in a highly bioavailable form that can be taken up by the body.

Bioavailability refers to the extent to which a nutrient or compound is absorbed and utilized by the body. Most traditional supplements have issues with bioavailability, meaning the ingredients therein are not effectively absorbed or utilized by the body. There are several factors that contribute to poor bioavailability in traditional supplements, including the formulations of the supplements, the quality of the ingredients, the particulate sizes of the ingredients, and the delivery method for the supplement.

Traditional supplements have specifically struggled to deliver effective antioxidants, such as glutathione, in a form enabling the glutathione to be absorbed and utilized by the body. In view of the foregoing, described herein are compositions, methods, and systems for improving free radical levels in a user and for treatment of oxidative stress in the user. The compositions, methods, and systems of the disclosure are also applicable for supporting the body's natural detoxification process and natural defense systems for combatting viruses, bacteria, heavy metal toxicity, radiation, certain medications, the process of aging, and so forth.

Specifically described herein are highly bioavailable compositions comprising nanoscale ingredients that can be absorbed and utilized by the body. As will be seen, the disclosure provides compositions, methods, and systems that can treat oxidative stress and improve the body's natural detoxification process and natural defense systems.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with respect to the following description and accompanying drawing where:

FIG. 1 is a schematic illustration of a process flow for preparing a nanoparticulated composition as described herein;

FIG. 2A illustrates the chemical structure of reduced glutathione (GSH) according to one implementation consistent with the teachings and principles of the disclosure;

FIG. 2B illustrates the chemical oxidation reaction of reduced glutathione (GSH) to oxidized glutathione (GSSG) according to one implementation consistent with the teachings and principles of the disclosure;

FIG. 3A is a schematic illustration of a traditional supplement ingredient failing to pass through a cellular wall due to particulate size;

FIG. 3B is a schematic illustration of a nanoparticulated ingredient passing through a cell wall due to reduced particulate size and supplement formulation;

FIG. 4 is a schematic flow chart diagram of a method for preparing a bioavailable composition as described herein; and

FIG. 5 is a schematic flow chart diagram of a method for calculating an effective amount of a composition to be provided to an animal to reduce oxidative stress in the animal.

### DETAILED DESCRIPTION

The disclosure extends to compositions comprising nanoscale ingredients with high bioavailability. Further described herein are systems, methods, and devices for manufacturing the nanoparticulated compositions described herein. The compositions described herein comprise bioavailable antioxidants, anti-inflammatory agents, and amino acids. Further described herein are systems and methods for improving physical performance, muscle development, strength, and lean body mass of an animal through administration of nanoparticulated ingredients.

The compositions described herein are formulated to provide effective amounts of bioavailable ingredients without providing an excess of those ingredients. Traditional supplements provide excess nutritional ingredients to a user, and the user only absorbs a small fraction of those nutritional ingredients. Thus, traditional supplements rely on the assumption that even if a high quantity of the nutritional ingredients are flushed by the user's system, the user's overall deficiency in those nutritional ingredients may be offset by providing the user with an excess.

The compositions described herein include nanoscale ingredients that exhibit increased uptake by the user's body when compared with traditional supplements. The interface between a supplement and the body takes place on the surface of the nutritional particle where most chemical and biological reactions occur. The compositions described herein increase the surface area of the supplement without increasing wasted volume by decreasing the size of the nutritional ingredients. Specifically, the compositions described herein include nutritional ingredients having a particle size of about 10 nm to about 200 nm. This significantly increases the bioavailability of the nutritional ingredients described herein.

Traditional supplements have a particle size of about 2 μm, and this produces a resultant surface area for the supplement of about 12 million nm². By contrast, the improved nanoscale ingredients described herein typically have a particle size from about 10 nm to about 80 nm, depending on the ingredient. In the compositions described herein, the total surface area of equal volumes of ingredients is typically from about 25 to about 200 times greater than traditional supplements. Thus, wherein a traditional supplement may have a total surface area of about 12 million nm², the compositions described herein will have a total surface area of about 120 million nm² to about 2.4 billion nm².

The compositions described herein include effective amounts of nutritional ingredients for reducing oxidative stress in a body. Oxidative stress, or elevated levels of free radicals in the body, is responsible for numerous health issues and is virtually impossible to avoid. Oxidative stress reflects an imbalance between the systemic manifestation of reactive oxygen species and the body's ability to readily detoxify the reactive intermediates or repair the resulting damage. Disturbances in the body's normal redox state of cells can cause toxic effects through the production of peroxides and free radicals that damage all components of a cell, including proteins, lipids, and DNA. Oxidative stress from oxidative metabolism causes base damage as well as strand breaks in DNA. Further, some reactive oxidative species function as cellular messengers in redox signaling. Thus, oxidative stress may cause disruptions in the body's normal mechanisms of cellular signaling.

Chemically, oxidative stress is associated with increased production of oxidizing species or a significant decrease in the effectiveness of antioxidant defenses, such as glutathione. Reduced glutathione (GSH) is often referred to as the body's primary antioxidant. Glutathione is composed of three amino acids, including cysteine, glycine, and glutamine, and glutathione can be found in virtually every cell of the body. The highest concentration of glutathione is typically found in the liver where it serves an important function in the body's natural detoxification process. This natural detoxification process is important for treating oxidative stress in the body and avoiding the production of damaging peroxides and free radicals.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

As used herein, “effective amount” means an amount of an ingredient or a component of the product that is nontoxic, but sufficient to provide the desired effect and performance at a reasonable benefit/risk ratio attending any dietary supplement or product. For example, an effective amount of a vitamin or mineral is an amount sufficient to prevent a deficiency thereof and to reduce the incidence of some adverse effects.

Unless defined otherwise, all technical and scientific terms used herein have the same meanings as commonly understood by one of ordinary skill in the art to which this disclosure pertains and belongs.

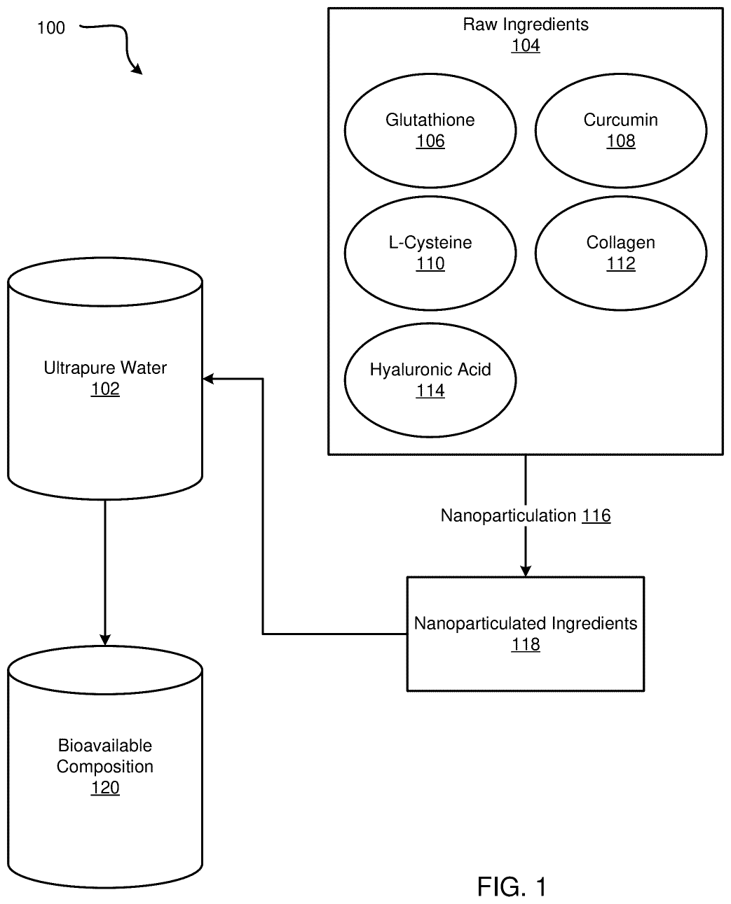

Referring now to the figures, FIG. 1 is a schematic diagram of a process flow 100 for preparing a bioavailable composition 120 as described herein. The process flow 100 includes selecting one or more raw ingredients 104, and then generating nanoparticulated ingredients 118 by causing the raw ingredients 104 to undergo a nanoparticulation 116 process. The process flow 100 additionally includes preparing ultrapure water 102 through an extensive purification process as described herein. The nanoparticulated ingredients 118 are added to the ultrapure water 102 to prepare the bioavailable composition 120.

The ultrapure water 102 (may be referred to as high-purity water or highly purified water (HPW)) is water that has been purified to a high degree, typically through one or more physical, chemical, or biological processes. The process flow 100 includes the preparation of the ultrapure water 102 prior to the nanoparticulated ingredients 118 being added to the ultrapure water 102. The purification process for ultrapure water 102 typically involves multiple stages, with each stage designed to remove specific impurities. The exact process may vary depending on the impurities present in the initial water. The process may include one or more of a pretreatment, reverse osmosis, deionization, distillation, and ultraviolet sterilization as described herein.

The preparation of the ultrapure water 102 typically includes a pretreatment stage, which includes filtering the initial water to remove large particles and sediment. The pretreatment stage may additionally include the use activated carbon absorption to remove organic matter. The preparation of the ultrapure water 102 typically includes a reverse osmosis stage, which uses a semi-permeable membrane to remove dissolved minerals, salts, and other contaminants. The preparation of the ultrapure water 102 may additionally include a deionization stage following the reverse osmosis, wherein the water is passed through an ion exchange resin to remove remaining ions and minerals. This stage may be performed using one or more of a cation exchange resin or an anion exchange resin. The preparation of the ultrapure water 102 may include a distillation stage, wherein the water is distilled to remove remaining contaminants and further purify the water. The distillation stage may include boiling the water and then collecting the resultant steam, which is then condensed back into a liquid stage. The preparation of the ultrapure water 102 may additionally include an ultraviolet (UV) sterilization and ozonation stage, wherein the water is treated with ultraviolet light or ozone to destroy remaining microorganisms.

During the purification process for the ultrapure water 102, the water is recirculated for continuous “polishing.” Polishing ultrapure water 102 refers to the process of removing remaining impurities or contaminants from the water that may still be present event after it has undergone various purification stages. The polishing process involves using additional purification methods, such as activated carbon filtration, ultraviolet light treatment, and reverse osmosis to remove remaining contaminants and ensure the water is as pure as possible.

The resultant ultrapure water 102 is free of impurities. The ultrapure water 102 may exhibit a resistivity of greater than 18 MΩ, a total organic carbon content of less than 10 parts per billion, an online dissolved oxygen measurement of 10 μg/L, an on-line particles measurement of less than 200 particles/L, and a microbiological or bacterial presence of less than 1 colony-forming unit/100 mL. The ultrapure water 102 may have a resistivity of at least 14 MΩ and may specifically have a resistivity between 16.4 MΩ to about 18.2 MΩ. The ultrapure water 102 is typically stored in a specially designed container or system to prevent contamination before the raw ingredients 104 are added.

The raw ingredients 104 are selected based on the desired result to be effectuated in the body. Typically, the raw ingredients 104 will include at least the glutathione 106. The glutathione 106 may include reduced glutathione (GSH) and/or oxidated glutathione (GSSG) as described herein. One or more of the raw ingredients 104 may be selected for the bioavailable composition 120, and effective amounts of each of the raw ingredients 104 will be determined based on the correct dosage rate for the body. Prior to adding the raw ingredients 104 to the ultrapure water 102, the raw ingredients undergo a nanoparticulation 116 process to generate the nanoparticulated ingredients 118.

The raw ingredients 104 may specifically include glutathione 106 and l-cysteine 110. The combination of glutathione 106 and l-cysteine 110, when nanoscale and disposed within an ultrapure water 102 solvent, exhibit unexpectedly good results at reducing oxidative stress in a user. The glutathione 106 and the l-cysteine 110, when used together, enable improved reduction of free radicals and overall improved reduction of oxidative stress in the body. The resultant compositions described herein may include additional nanoscale ingredients as described herein, including, for example, the curcumin 108, collagen 112, and hyaluronic acid 114 illustrated in FIG. 1.

The nanoparticulation 116 process is executed to reduce the particle sizes of the raw ingredients 104. In some cases, the nanoparticulation 116 process includes one or more of micronization or the use of high sheer, high speed homogenizers to reduce particle size.

For water-soluble ingredients, the nanoparticulation 116 process may be skipped because those water-soluble ingredients will form nano-sized particles when dissolved within the ultrapure water 102. Thus, the nanoparticulation 116 process is ingredient-dependent, where some ingredients will undergo a pre-solvent nanoparticulation 116 process involving, for example, micronization through the use of high sheer, high speed homogenizers. Other water-soluble raw ingredients 104 will not undergo a pre-solvent nanoparticulation 116 process and will instead form nano-sized particles when dissolved within the ultrapure water 102.

High shear, high-speed homogenizers are machines configured to mix, disperse, and emulsify fluids or powders. These homogenizers may be used to prepare the raw ingredients 104 prior to addition to the ultrapure water 102 by using high shear forces to break down particles and mix them evenly into a fluid medium. The high shear forces are generated by a rapidly rotating rotor and stator, which creates intense turbulence and shear in the fluid. The high speed of the roto can range from about 5,000 RPM to about 50,000 RPM depending on the specific application. The homogenization process can be used to achieve a variety of outcomes, including particle size reduction, emulsification of immiscible fluids, and dispersion of powders in liquids.

Micronization is a process that involves reducing the size of particles to micron or sub-micron levels. The process of micronization typically involves two stages, namely particle size reduction and particle size classification. Particle size reduction can be achieved through a variety of methods, including mechanical milling, jet milling, and spray drying. Mechanical milling involves using a mechanical force to break down particles. Jet milling uses high-pressure air or gas to grind particles into smaller sizes. Spray drying involves atomizing a liquid solution or suspension into a fine mist and drying the particles with hot air. Particle size classification involves separating the micronized particles into different size ranges, typically using a sieve or air classifier. This helps ensure the final raw ingredient 104 has a consistent size distribution.

The raw ingredients 104 illustrated in FIG. 1 are exemplary only, and the resultant composition may include any combination of the raw ingredients 104 illustrated in FIG. 1 and may include other raw ingredients not shown. The raw ingredients 104 added to the ultrapure water 102 may include one or more of glutathione 106, curcumin 108, L-cysteine 110, collagen 112, and hyaluronic acid 114, calcium ascorbate, methylsulfonylmethane, hydroxyproline, arginine, valine, proline, glycine, and others. The raw ingredients 104 added to the ultrapure water 102 may specifically include other botanical ingredients or other naturally occurring ingredients.

Reduced glutathione (GSH) 106 is a key component of the body's natural defense system. Glutathione 106 can be depleted by compositions or processes that are associated with free-radical damage, including for example, viruses, bacteria, heavy metal toxicity, radiation, certain medications, and the process of aging. The depletion of glutathione 106 is associated with lower immune function, increased vulnerability to infection, and a reduction in the liver's ability to detoxify the body. As the generation of free radicals exceeds the body's ability to neutralize and eliminate them, oxidative stress occurs. A primary function of glutathione 106 is to alleviate oxidative stress.

Glutathione 106 is under tight homeostatic control both intracellularly and extracellularly. A dynamic balance is maintained between the synthesis of reduced glutathione 106, its recycling from oxidized glutathione 106, and its utilization. Reduced glutathione 106 can be depleted by direct attack by free radicals and other oxidative agents. The homeostatic glutathione redox cycle attempts to keep reduced glutathione 106 repleted as it is being consumed. Equivalents of reduced glutathione 106 circulate in the blood predominately as cystine (i.e., the oxidized and more stable form of cysteine.) Cells import cystine from the blood, reconvert it to cysteine, and form it to synthesize reduced glutathione 106. Conversely, inside the cell, reduced glutathione 106 assists in re-reducing oxidized forms of other antioxidants such as ascorbate and alpha-tocopherol.

The compositions described herein include nanoparticulated and bioavailable glutathione. Specifically, the compositions described herein include an effective amount of bioavailable glutathione for reducing oxidative stress in the body, and this may further result in decreasing inflammation, reducing joint discomfort, improving the immune system, increasing energy, improving muscular stamina and endurance, decreasing exercise recovery times, improving mental clarity and focus, improving sleep, delaying the effects of aging, removing chemical toxins from cells, and improving oxygen transfer.

Curcumin 108 is a naturally occurring chemical compound found in turmeric. Curcumin 108 is a potent anti-inflammatory and antioxidant agent, and curcumin 108 can be beneficial for people with conditions such as arthritis, heart disease, and cancer. Curcumin 108 is also associated with improving brain function and reducing the risk of age-related cognitive decline. In traditional supplements, the bioavailability of curcumin 108 is relatively low, meaning the body cannot absorb it very well. To address this, the compositions described herein include nanoparticulated curcumin 108 within an ultrapure water 102 solvent, and this exhibits unexpectedly good results in improving the bioavailability of curcumin 108.

L-cysteine 110 is an amino acid that contains a thiol group in its side chain. L-cysteine 110 is found in high-protein foods such as meat, poultry, eggs, and dairy products, as well as in some plant-based sources such as legumes and grains. L-cysteine 110 has several important functions in the body, namely, it serves as a precursor to glutathione 106, which is a powerful antioxidant that helps protect cells from damage caused by free radicals. L-cysteine 110 is also involved in the synthesis of proteins, and is a key component of keratin, which is a protein found in hair, skin, and nails.

Collagen 112 is a protein found in the skin, bones, muscles, tendons, and other connective tissues in the body. Collagen 112 is the most abundant protein in the human body and is essential for maintaining the strength of structure of these tissues. Collagen 112 is made up of a unique combination of amino acids, including glycine, proline, and hydroxyproline. As bodies age, they produce less collagen 112, and this can lead to wrinkles, joint pain, and other signs of aging. Traditional collagen supplements exhibit relatively low bioavailability, meaning the collagen 112 molecules are not effectively taken up and used by the body. However, according to the composition preparations described herein, the bioavailable composition 120 within ultrapure water 102 exhibits unexpectedly good results in improving the bioavailability of collagen 112.

Hyaluronic acid 114 is a naturally occurring polysaccharide found in the body, and it serves as a key component of connective tissue found in the skin, joints, and eyes. Hyaluronic acid 114 is known for its ability to hold water and plays a significant role in maintaining hydration and elasticity in the skin. Hyaluronic acid 114 is also involved in lubricating and cushioning joints and is found in the vitreous humor of the eye, where it helps maintain the shape of the eye and protect the retina.

The combination of the ultrapure water 102 and the nanoparticulated ingredients 118 exhibits unexpectedly good results in generating a bioavailable composition 120. The bioavailable composition 120 exhibits increased bioavailability of the raw ingredients 104 when compared with traditional supplements. The process flow 100 maintains the natural properties of ultrapure water 102 to provide unique water cluster protection of nano-sized particles, and this increases the bioavailability and transference of the ingredients into organic cells.

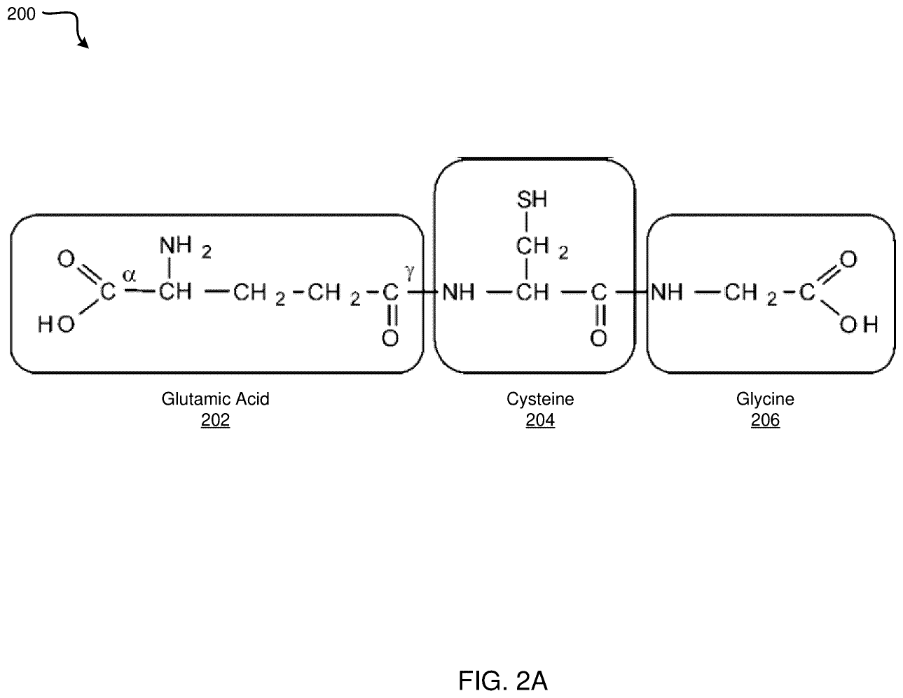

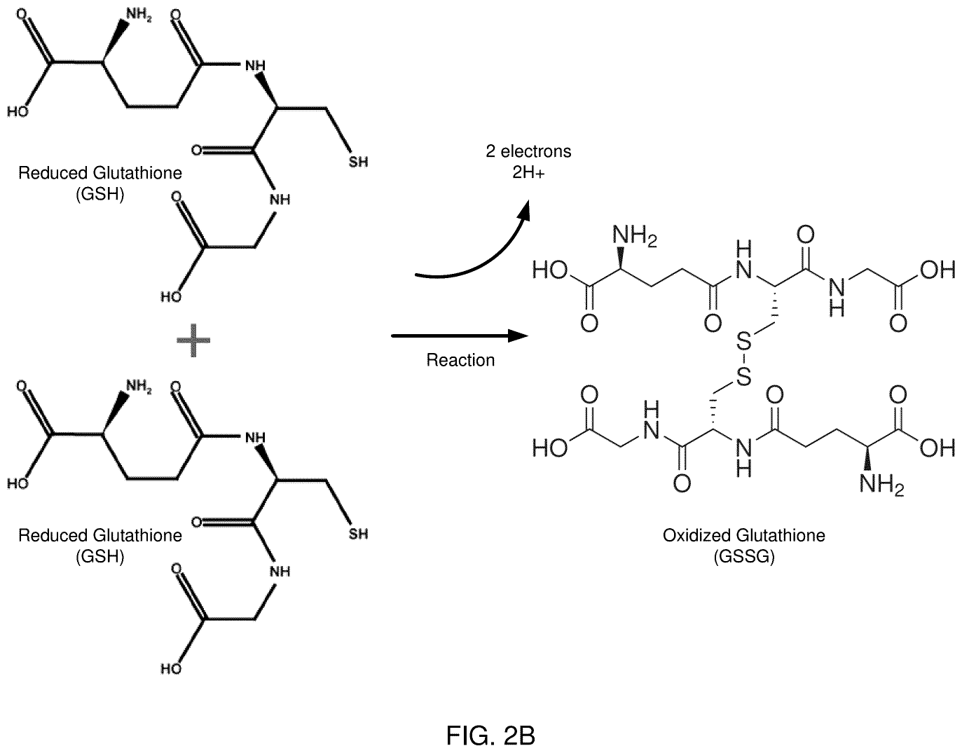

FIG. 2A illustrates the chemical structure 200 of reduced glutathione 106. As illustrated, reduced glutathione 106 comprises glutamic acid 202, cysteine 204, and glycine 206. Reduced glutathione (GSH) is a linear tripeptide of L-glutamine, L-cysteine, and glycine. Reduced glutathione 106 is technically referred to as N-L-gamma-glutamyl-cysteinyl glycine or L-glutathione. The molecule includes a sulfhydryl (SH) group on the cysteinyl portion that accounts for its strong electron-donating character that enables glutathione 106 to be effective in reducing oxidative stress in the body. Glutathione 106 is oxidized as an electron is lost, and two such molecules (with a lost electron) become linked or dimerized by a disulfide bridge. The dimerized molecules form glutathione 106 disulfide or oxidized glutathione (GSSG). The linkage is reversible upon re-reduction.

The synthesis of reduced glutathione 106 includes two closely linked and enzymatically controlled reactions that utilize adenosine triphosphate (ATP). First, cysteine and glutamate are combined by gamma-glutamyl cysteinyl synthetase. Second, reduced glutathione 106 synthetase combines gamma-glutamylcysteine with glycine to generate reduced glutathione 106. As the levels of reduced glutathione 106 rise, the processes are self-limited against further production of reduced glutathione 106. Otherwise, cysteine availability is usually rate-limiting. Fasting, protein-energy malnutrition, and other dietary amino acid deficiencies limit the synthesis of reduced glutathione 106. The recycling of reduced glutathione 106 is catalyzed by glutathione disulfide reductase, which uses reducing equivalents from nicotinamide adenine dinucleotide phosphate (NADPH) to reconvert oxidized glutathione to glutathione disulfide. The reducing power of ascorbate helps conserve systemic reduced glutathione 106.

FIG. 2B illustrates the oxidation reaction of reduced glutathione (GSH) to oxidized glutathione (GSSG). The reaction produces two electrons and two protons as illustrated. In the process of neutralizing free radicals in the body, reduced glutathione (GSH) can become oxidized to its disulfide from (GSSG). The oxidation of GSH to GSSH and its subsequent reduction back to GSH plays a significant role in maintaining the balance between oxidative stress and antioxidant defenses in the body.

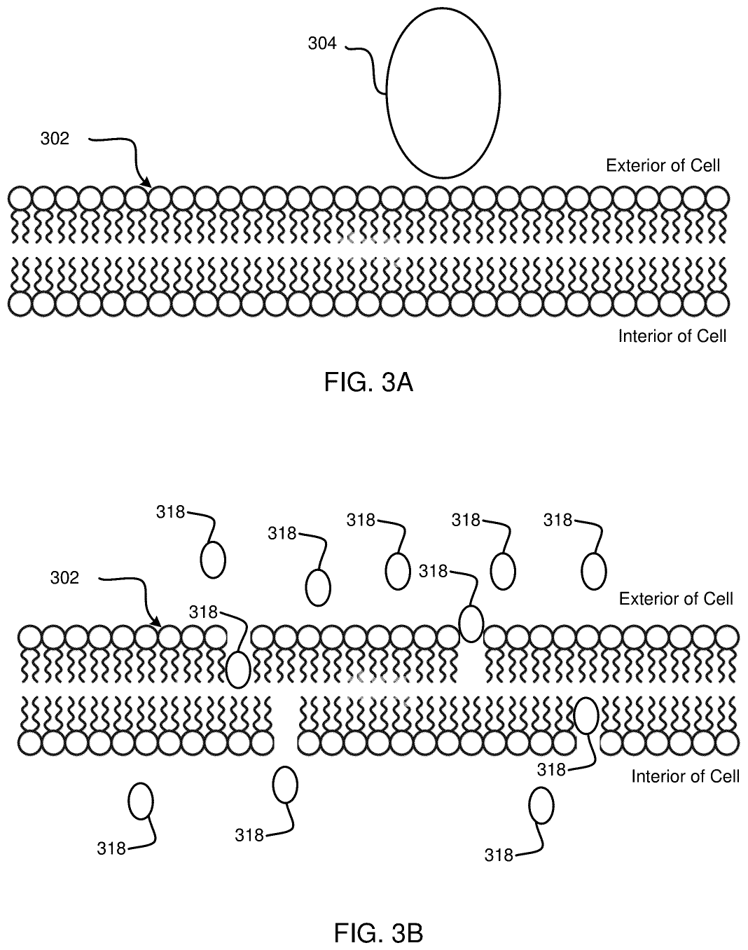

FIGS. 3A-3B are schematic illustration depicting the unexpectedly good results achieved by the bioavailable composition 120 described herein. FIG. 3A is a schematic illustration depicting a traditional non-nanoparticulated raw ingredient 304, without an ultrapure water 102 solvent, attempting to cross the bilayer 302 of a cell wall. FIG. 3B is a schematic illustration depicting the bioavailable composition 120, which comprises the nanoparticulated ingredients 318 within an ultrapure water 102 solvent, readily crossing the bilayer 302 an entering the interior of the cell.

As shown in FIG. 3A, the bioavailability of the non-nanoparticulated raw ingredient 302 is low because the raw ingredient 304 is too large to cross the bilayer 302 and cross from the exterior of the cell into the interior of the cell. Thus, the raw ingredient 302 cannot be taken up and utilized by the cell.

By contrast, as shown in FIG. 3B, the bioavailability of the nanoparticulated ingredients 318 is high because the nanoparticulated ingredients 318 are sized such that they can readily cross the bilayer 302 of the cell. The nanoparticulated ingredients 318 cross the bilayer 302 to pass from the exterior of the cell into the interior of the cell. When the nanoparticulated ingredients 318 reach the interior of the cell, they can be taken up and utilized by the body.

The combination of the ultrapure water 102 solvent and the nanoparticulated ingredients 118 enables the unexpectedly good results illustrated in FIG. 3B. The ultrapure water 102 is highly reactive and serves to maintain the nanoparticulated ingredients 118 in their nanoparticulated state, with a particle size from about 10 nm to about 150 nm. The nano size of the nanoparticulated ingredients 118 enables easy uptake by the cell because the nanoparticulated ingredients 118 can pass through the phospholipid bilayer of the cell wall.

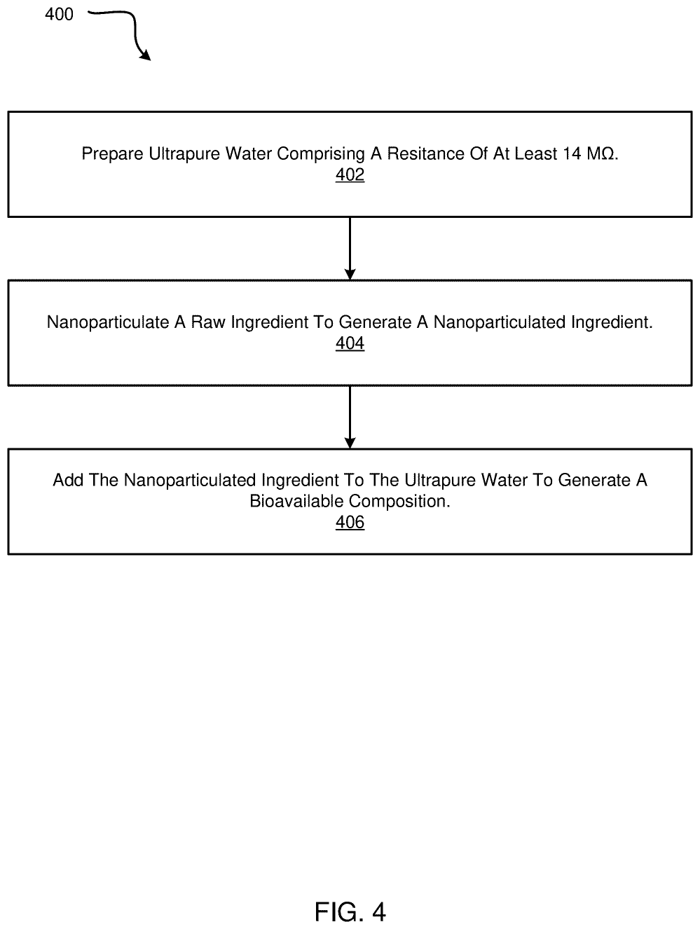

FIG. 4 is a schematic flow chart diagram of a method 400 for preparing a bioavailable composition 120 as described herein. The method 400 begins with preparing at 402 ultrapure water 102 such that the resultant ultrapure water 102 comprises a resistance of at least 14 MΩ. The process of preparing the ultrapure water 102 may include any of the processes described herein, including, mechanical filtering, activated carbon absorption, reverse osmosis, deionization, distillation, ultraviolet sterilization, and ozone-based sterilization. The process of preparing the ultrapure water 102 may include any other processes known in the art.

The method 400 continues with nanoparticulating at 404 a raw ingredient 104 to generate a nanoparticulated ingredient 118. The resultant nanoparticulated ingredient 118 has an average particle size of less than 500 nm and may specifically have an average particle size from about 10 nm to about 150 nm. The process of nanoparticulating the raw ingredient may include any of the processes described herein, including micronization and undergoing high-speed, high-sheer homogenization. The raw ingredient may include one or more of calcium ascorbate, reduced glutathione (GSH), oxidized glutathione (GSSG), 1-glutathione, l-cysteine, curcumin, arginine, valine, proline, glycine, hyaluronic acid, methylsulfonylmethane, collagen, or hydroxyproline. The raw ingredient added to the ultrapure water may additionally or alternatively include other botanical ingredients or other naturally occurring ingredients. The nanoparticulated ingredient may be prepared to comprise a solid form and specifically to comprise a fine powder form prior to being added to the ultrapure water.

The method 400 continues and the nanoparticulated ingredient is added to the ultrapure water at 406 to generate the bioavailable composition 120. This process may include combining one or more nanoparticulated ingredients and then adding the combination of nanoparticulated ingredients to a small amount of ultrapure water to generate a slurry comprising a relatively thick viscosity. The reactive ultrapure water and nanoparticulated ingredient slurry may then be combined within a specialized reaction vessel. The slurry (comprising the combination of nanoparticulated ingredients and the small amount of the ultrapure water) may then be added to a greater quantity of the ultrapure water.

The greater quantity of the ultrapure water may be subject to disruption of its resting state prior to adding the slurry. This disruption process may include subjecting the ultrapure water to agitation, bubbling, stirring, churning, spinning, flow topology, or another similar process.

Prior to adding the slurry to the disrupted ultrapure water, the slurry undergoes high-pressure (at least 250 psi) injection in a patterned injection. The injection volume is relatively small and may be less than 0.5 mL of slurry per cycle, with frequency of cycles from about 60 to about 120 cycles per minute.

Further prior to adding the slurry to the disrupted ultrapure water, additional outside energy is applied to the slurry. The additional outside energy includes one or more of electricity, magnetic flux, electromagnetic energy, infrared radiation, ultraviolet rays, X-rays, gamma rays, pico laser bursts, vibratory oscillations, or other external coalescing force. The slurry is then added to the disrupted ultrapure water. The resulting bioavailable composition is stable aqueous composition comprising water and one or more nanoparticulated ingredients. The method 400 may further include adding one or more preservatives, flavors, or pH adjustors to the bioavailable composition.

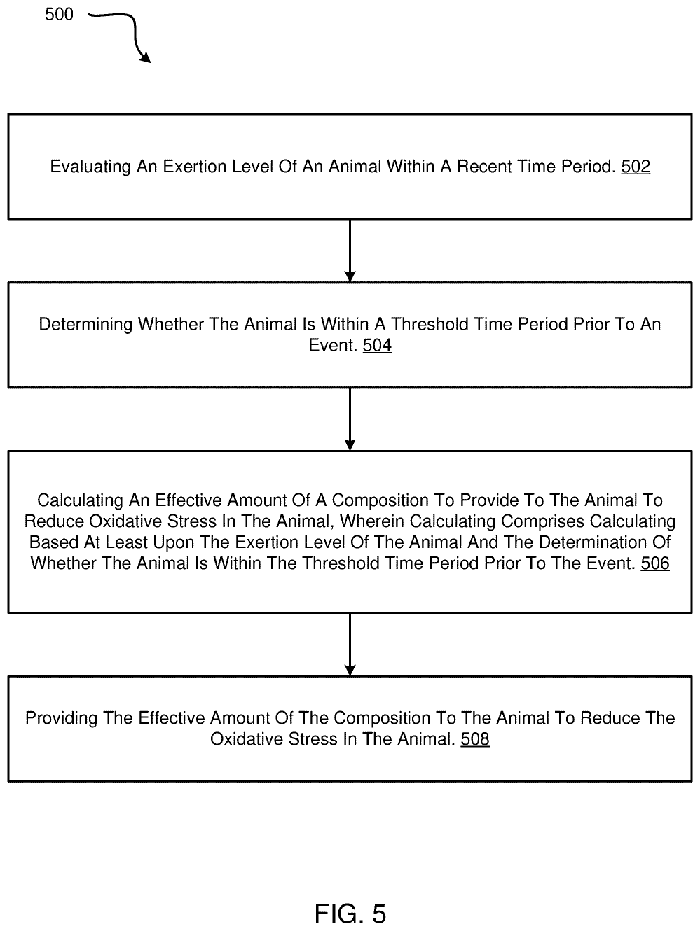

FIG. 5 is a schematic flow chart diagram of a method 500 for calculating an effective amount of a composition to be provided to an animal to reduce oxidative stress in the animal. The method 500 may be performed to calculate an effective amount of any of the compositions described herein, including, for example, the compositions comprises a nanoparticulated ingredient that may include one or more of nanoparticulated glutathione (GSH), nanoparticulated glutathione (GSSG), nanoparticulated curcumin, nanoparticulated l-cysteine, nanoparticulated collagen, nanoparticulated hyaluronic acid, nanoparticulated calcium ascorbate, nanoparticulated methylsulfonylmethane, nanoparticulated hydroxyproline, nanoparticulated arginine, nanoparticulated valine, nanoparticulated proline, nanoparticulated glycine, and so forth.

The method 500 may be performed to determine the effective amount of the composition for reducing oxidative stress in an animal. In some cases, the animal is a human user. In other cases, the animal is a non-human animal, and may specifically include, for example, a bovine, feline, canine, junglefowl, Equidae, or another animal. The method 500 may be performed to prepare an animal for a certain purpose and may specifically be performed to improve one or more of the physical performance, muscle development, strength, lean body mass, or other characteristic of the animal.

The method 500 includes evaluating at 502 an exertion level of an animal within a recent time period. The recent time period may include, for example, the past one hour, the past day, the past two days, the past three days, the past week, the past month, the past multiple months, the past year, and so forth. The boundaries of the recent time period may vary depending on the type of animal, age of the animal, history of the animal, goals for improving the animal, and so forth. The exertion level may be determined based on, for example, an estimated quantity of energy utilized by the animal per day, a duration of time the animal is training or exercising per day, and so forth.

The method 500 includes determining at 504 whether the animal is within a threshold time period prior to an event. The event may include any suitable event and may vary depending on the type of animal. For example, if the animal is a human, then the event may include one or more of an athletic event, a sporting event, a performance, a bodybuilding show, a competition, a planned medical procedure, and so forth. Further for example, if the animal is within the Equidae taxonomic family of horses, then the event may include one or more of a race, equestrian show, showjumping competition, aesthetics show, and so forth. The type of event may vary greatly depending on the type of animal.

The threshold time period prior to the event may also vary depending upon, for example, the type of event, type of animal, age of the animal, health of the animal, current progress of the animal, and so forth. The threshold time period may refer to a time period when the animal is recommended to receive an increased dose of the composition to more effectively prepare for the event.

The method 500 includes calculating at 506 an effective amount of a composition to provide to the animal to reduce oxidative stress in the animal. The calculating may be performed based at least upon the exertion level of the animal within the recent time period and further upon the determination of whether the animal is within a threshold time period prior to the event.

The effective amount of the composition will vary depending upon the exertion level of the animal within the recent time period. If the animal is undergoing above average levels of exertion, then the animal will benefit from a greater dose to reduce levels of oxidative stress in the animal. If the animal is recovering from a medical procedure, then the animal will benefit from a greater dose to reduce levels of oxidative stress in the animal.

The effective amount of the composition will vary depending upon whether the animal is within the threshold time period prior to the event. If the animal is within the threshold time period, then the animal may benefit from an increased dose to aid the animal in preparing to succeed in the event. If the animal is outside the threshold time period, then the animal may receive a reduced benefit from an increased dose.

The effective amount of the composition will further depend on other factors such as, for example, the type of animal, the weight of the animal, the age of the animal, the amount of oxidative stress experienced by the animal, the diet of the animal, and so forth.

The method 500 includes providing at 508 the effective amount of the composition to the animal to reduce the oxidative stress in the animal. The composition may include any of the compositions described herein and may specifically include any of the composition described in connection with any of Tables 1-17 or Examples 1-62, below.

### EXAMPLES

The following examples pertain to further embodiments.

Table 1, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 5 vol % |
| Glutathione | 1 vol % |
| L-cysteine | 20 vol % |
| Curcumin | 74 vol % |

Table 2, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 10 vol % |
| Glutathione | 2 vol % |
| L-cysteine | 20 vol % |
| Curcumin | 68 vol % |

Table 3, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 7 vol % |
| Glutathione | 5 vol % |
| L-cysteine | 30 vol % |
| Curcumin | 58 vol % |

Table 4, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 6 vol % |
| Glutathione | 4 vol % |
| L-cysteine | 70 vol % |
| Curcumin | 20 vol % |

Table 5, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 10 vol % |
| Glutathione | 2 vol % |
| L-cysteine | 64 vol % |
| Curcumin | 24 vol % |

Table 6, below, illustrates an example formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | 8 vol % |
| Glutathione | 4 vol % |
| L-cysteine | 59 vol % |
| Curcumin | 29 vol % |

Table 7, below, illustrates example ranges for a formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | from about 5 vol % to about 10 vol % |
| Glutathione | from about 10 vol % to about 70 vol % |
| L-cysteine | from about 20 vol % to about 70 vol % |
| Curcumin | from about 1 vol % to about 5 vol % |

Table 8, below, illustrates example ranges for a formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | from about 5 vol % to about 10 vol % |
| Glutathione and L-cysteine | from about 80 vol % to about 94 vol % |
| Curcumin | from about 1 vol % to about 5 vol % |

Table 9, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 5 | vol % |
| L-glutathione | 30 | vol % |
| L-cysteine | 25 | vol % |
| Curcumin | 1 | vol % |
| Arginine | 1 | vol % |
| Valine | 1 | vol % |
| Proline | 4 | vol % |
| Glycine | 3 | vol % |
| Hyaluronic acid | 15 | vol % |
| Methylsulfonylmethane | 10 | vol % |
| Collagen | 2 | vol % |
| Hydroyproline | 3 | vol % |

Table 10, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 8 | vol % |
| L-glutathione | 25 | vol % |
| L-cysteine | 35 | vol % |
| Curcumin | 1 | vol % |
| Arginine | 1 | vol % |
| Valine | 1 | vol % |
| Proline | 4 | vol % |
| Glycine | 3 | vol % |
| Hyaluronic acid | 8 | vol % |
| Methylsulfonylmethane | 10 | vol % |
| Collagen | 2 | vol % |
| Hydroyproline | 2 | vol % |

Table 11, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 5 | vol % |
| L-glutathione | 35 | vol % |
| L-cysteine | 35 | vol % |
| Curcumin | 1 | vol % |
| Arginine | 1 | vol % |
| Valine | 1 | vol % |
| Proline | 3 | vol % |
| Glycine | 2 | vol % |
| Hyaluronic acid | 5 | vol % |
| Methylsulfonylmethane | 10 | vol % |
| Collagen | 1 | vol % |
| Hydroyproline | 1 | vol % |

Table 12, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 5 | vol % |
| L-glutathione | 20 | vol % |
| L-cysteine | 35 | vol % |
| Curcumin | 1 | vol % |
| Arginine | 1 | vol % |
| Valine | 1 | vol % |
| Proline | 2 | vol % |
| Glycine | 1 | vol % |
| Hyaluronic acid | 12 | vol % |
| Methylsulfonylmethane | 18 | vol % |
| Collagen | 2 | vol % |
| Hydroyproline | 2 | vol % |

Table 13, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 6 | vol % |
| L-glutathione | 27 | vol % |
| L-cysteine | 31 | vol % |
| Curcumin | 4 | vol % |
| Arginine | 3 | vol % |
| Valine | 4 | vol % |
| Proline | 4 | vol % |
| Glycine | 3 | vol % |
| Hyaluronic acid | 8 | vol % |
| Methylsulfonylmethane | 7 | vol % |
| Collagen | 1 | vol % |
| Hydroyproline | 2 | vol % |

Table 14, below, illustrates an example formulation of the bioavailable compositions 120 described herein.

| | Volume Percentage of Nanoparticulated | |
|---|---|---|
| Nanoparticulated | Ingredient in Formulation Prior to | |
| Ingredient | Addition to Ultrapure Water Solvent | |
| Calcium ascorbate | 8 | vol % |
| L-glutathione | 22 | vol % |
| L-cysteine | 34 | vol % |
| Curcumin | 5 | vol % |
| Arginine | 2 | vol % |
| Valine | 5 | vol % |
| Proline | 3 | vol % |
| Glycine | 6 | vol % |
| Hyaluronic acid | 5 | vol % |
| Methylsulfonylmethane | 7 | vol % |
| Collagen | 1 | vol % |
| Hydroyproline | 2 | vol % |

Table 15, below, illustrates example ranges for a formulation of the bioavailable compositions 120 described herein.

| | Volume Percentage of Nanoparticulated |
|---|---|
| Nanoparticulated | Ingredient in Formulation Prior to |
| Ingredient | Addition to Ultrapure Water Solvent |
| Calcium ascorbate | from about 3 vol % to about 8 vol % |
| L-glutathione | from about 20 vol % to about 35 vol % |
| L-cysteine | from about 20 vol % to about 35 vol % |
| Curcumin | from about 1 vol % to about 5 vol % |
| Arginine | from about 1 vol % to about 5 vol % |
| Valine | from about 1 vol % to about 5 vol % |
| Proline | from about 1 vol % to about 7 vol % |
| Glycine | from about 1 vol % to about 6 vol % |
| Hyaluronic acid | from about 5 vol % to about 15 vol % |
| Methylsulfonylmethane | from about 10 vol % to about 20 vol % |
| Collagen | from about 1 vol % to about 5 vol % |
| Hydroyproline | from about 1 vol % to about 5 vol % |

Table 16, below, illustrates example ranges for a formulation of the bioavailable compositions 120 described herein.

| Nanoparticulated | Molar Percentage of Nanoparticulated |
|---|---|
| Ingredient | Ingredient in Formulation |
| Calcium ascorbate | from about 0.052 mol % to about 0.31 mol % |
| L-glutathione | from about 0.18 mol % to about 1.1 mol % |
| L-cysteine | from about 0.071 mol % to about 0.21 mol % |
| Curcumin | from about 0.01 mol % to about 0.4 mol % |
| Ultrapure water | from about 99.7 mol % to about 97.5 mol % |

Table 17, below, illustrates example ranges for a formulation of the bioavailable compositions 120 described herein.

| Nanoparticulated | Molar Percentage of Nanoparticulated |
|---|---|
| Ingredient | Ingredient in Formulation |
| Calcium ascorbate | from about 0.052 mol % to about 0.31 mol % |
| L-glutathione | from about 0.18 mol % to about 1.1 mol % |
| L-cysteine | from about 0.071 mol % to about 0.21 mol % |
| Curcumin | from about 0.01 mol % to about 0.4 mol % |
| Arginine | from about 0 mol % to about 0.015 mol % |
| Valine | from about 0 mol % to about 0.01 mol % |
| Proline | from about 0 mol % to about 0.014 mol % |
| Glycine | from about 0 mol % to about 0.01 mol % |
| Hyaluronic acid | from about 0 mol % to about 0.72 mol % |
| Methylsulfonylmethane | from about 0 mol % to about 0.03 mol % |
| Collagen | from about 0 mol % to about 0.01 mol % |
| Hydroxyproline | from about 0 mol % to about 0.01 mol % |
| Ultrapure water | from about 99.7 mol % to about 97.5 mol % |

The bioavailable composition 120 described herein may be formulated such that the nanoparticulated ingredients 118 (prior to addition to the ultrapure water 102) comprise from about 80 vol % to about 95 vol % the combination of the glutathione 106 and the l-cysteine 110. The volumetric ratio of the glutathione 106 to the l-cysteine may be from about 1:3 to about 3:1. In an implementation, the volumetric ratio of the glutathione 106 to the l-cysteine is about 1:3. In an implementation, the volumetric ratio of the glutathione 106 to the l-cysteine is about 3:1. In an implementation, the volumetric ratio of the glutathione 106 to the l-cysteine is about 1:2.5. the volumetric ratio of the glutathione 106 to the l-cysteine is about 2.5:1. the volumetric ratio of the glutathione 106 to the l-cysteine is about 1:2. the volumetric ratio of the glutathione 106 to the 1-cysteine is about 2:1. the volumetric ratio of the glutathione 106 to the l-cysteine is about 1:1.

In some embodiments, the bioavailable composition 120 may comprise from about 92 molar percent (mol %) to about 99.8 mol % of the ultrapure water 102.

The foregoing percentages, concentrations, and ratios are presented by example only and are not intended to be exhaustive or to limit the disclosure to the precise percentages, concentrations, and ratios disclosed. It should be appreciated that each value that falls within a disclosed range is disclosed as if it were individually disclosed as set forth herein. For example, a range indicating a weight percent from about 8% to about 14% additionally includes ranges beginning or ending with all values within that range, including for example a range beginning at 8.1%, 8.2%, 8.3%, and so forth.

Example 1 is a composition. The composition includes a nanoparticulated ingredient selected from a list comprising glutathione, l-cysteine, and curcumin. The composition comprises a water solvent, wherein the water solvent comprises ultrapure water prior to addition of the nanoparticulated ingredient.

Example 2 is a composition as in Example 1, wherein the ultrapure water comprises a resistance of at least 14 MΩ prior to the addition of the nanoparticulated ingredient.

Example 3 is a composition as in any of Examples 1-2, wherein the ultrapure water comprises a resistance of at least 16 MΩ prior to the addition of the nanoparticulated ingredient.

Example 4 is a composition as in any of Examples 1-3, wherein the ultrapure water comprises a resistance of at least 17 MΩ prior to the addition of the nanoparticulated ingredient.

Example 5 is a composition as in any of Examples 1-4, wherein the nanoparticulated ingredient comprises a particle size from about 10 nm to about 100 nm.

Example 6 is a composition as in any of Examples 1-5, wherein the nanoparticulated ingredient comprises a particle size less than 500 nm.

Example 7 is a composition as in any of Examples 1-6, wherein the nanoparticulated ingredient is reduced glutathione (GSH), and wherein the reduced glutathione (GSH) is added to the ultrapure water in a solid form.

Example 8 is a composition as in any of Examples 1-7, wherein the nanoparticulated ingredient comprises each of the glutathione, the l-cysteine, and the curcumin.

Example 9 is a composition as in any of Examples 1-8, further comprising l-cysteine.

Example 10 is a composition as in any of Examples 1-9, further comprising arginine.

Example 11 is a composition as in any of Examples 1-10 further comprising valine.

Example 12 is a composition as in any of Examples 1-11, further comprising proline.

Example 13 is a composition as in any of Examples 1-12, further comprising glycine.

Example 14 is a composition as in any of Examples 1-13, further comprising hyaluronic acid.

Example 15 is a composition as in any of Examples 1-14, further comprising methylsulfonylmethane.

Example 16 is a composition as in any of Examples 1-15, further comprising collagen.

Example 17 is a composition as in any of Examples 1-16, further comprising hydroxyproline.

Example 18 is a composition as in any of Examples 1-17, wherein the composition is prepared for oral consumption.

Example 19 is a composition as in any of Examples 1-18, wherein the composition is prepared for topical administration.

Example 20 is a composition as in any of Examples 1-19, wherein the composition is prepared for one or more of intravenous or intramuscular administration.

Example 21 is a method. The method includes preparing ultrapure water comprising a resistance of at least 14 MΩ. The method includes nanoparticulating a raw ingredient to generate a nanoparticulated ingredient. The method includes adding the nanoparticulated ingredient to the ultrapure water to generate a bioavailable composition.

Example 22 is a method as in Example 21, wherein the ultrapure water comprises a resistance of at least 16 MΩ.

Example 23 is a method as in any of Examples 21-22, wherein the ultrapure water comprises a resistance of at least 17 MΩ.

Example 24 is a method as in any of Examples 21-23, wherein preparing the ultrapure water comprises filtering water to remove particles comprising a size greater than an initial particle threshold, and further to remove sediment.

Example 25 is a method as in any of Examples 21-24, wherein preparing the ultrapure water comprises processing water with an activated carbon absorption process.

Example 26 is a method as in any of Examples 21-25, wherein preparing the ultrapure water comprises processing water with reverse osmosis.

Example 27 is a method as in any of Examples 21-26, wherein preparing the ultrapure water comprises processing water with deionization.

Example 28 is a method as in any of Examples 21-27, wherein preparing the ultrapure water comprises processing water with distillation.

Example 29 is a method as in any of Examples 21-28, wherein preparing the ultrapure water comprises processing water with ultraviolet or ozone sterilization.

Example 30 is a method as in any of Examples 21-29, wherein the nanoparticulated ingredient comprises nanoparticulated glutathione.

Example 31 is a method as in any of Examples 21-30, wherein the nanoparticulated ingredient comprises nanoparticulated reduced glutathione (GSH).

Example 32 is a method as in any of Examples 21-31, wherein the nanoparticulated ingredient comprises nanoparticulated 1-glutathione.

Example 33 is a method as in any of Examples 21-32, wherein the nanoparticulated ingredient comprises l-cysteine.

Example 34 is a method as in any of Examples 21-33, wherein the nanoparticulated ingredient comprises collagen.

Example 35 is a method as in any of Examples 21-34, wherein the nanoparticulated ingredient comprises curcumin.

Example 36 is a method as in any of Examples 21-35, further comprising adding one or more of calcium ascorbate, arginine, valine, proline, glycine, hyaluronic acid, methylsulfonylmethane, collagen, or hydroxyproline to the bioavailable composition. The bioavailable composition may additionally or alternatively include other botanical ingredients or other naturally occurring ingredients.

Example 37 is a method as in any of Examples 21-36, wherein nanoparticulating the raw ingredient comprises processing the raw ingredient with a high-speed, high-sheer homogenization process.

Example 38 is a method as in any of Examples 21-37, wherein nanoparticulating the raw ingredient comprises processing the raw ingredient with a micronization process.

Example 39 is a method as in any of Examples 21-38, wherein adding the nanoparticulated ingredient to the ultrapure water comprises preparing a slurry comprising the nanoparticulated ingredient and the ultrapure water.

Example 40 is a method as in any of Examples 21-39, further comprising processing the bioavailable composition with one or more of agitation, bubbling, stirring, churning, spinning, or flow topology.

Example 41 is a composition. The composition includes a nanoparticulated ingredient selected from a list comprising glutathione, l-cysteine, and curcumin. The composition includes a water solvent, wherein the water solvent comprises ultrapure water prior to addition of the nanoparticulated ingredient.

Example 42 is a composition as in Example 41, wherein the ultrapure water comprises a resistance of at least 14 megaohms prior to the addition of the nanoparticulated ingredient.

Example 43 is a composition as in any of Examples 41-42, wherein the nanoparticulated ingredient comprises a particle size from about 10 nanometers to about 100 nanometers.

Example 44 is a composition as in any of Examples 41-43, wherein the nanoparticulated ingredient comprises a particle size less than 500 nanometers.

Example 45 is a composition as in any of Examples 41-44, wherein the glutathione of the nanoparticulated ingredient is reduced glutathione, and wherein the reduced glutathione is added to the ultrapure water in a solid form.

Example 46 is a composition as in any of Examples 41-45, wherein the nanoparticulated ingredient comprises each of the glutathione, the l-cysteine, and the curcumin.

Example 47 is a composition as in any of Examples 41-46, wherein the composition comprises an effective amount of the nanoparticulated ingredient to reduce oxidative stress in a body.

Example 48 is a composition as in any of Examples 41-47, wherein the nanoparticulated ingredient of the composition comprises: an effective amount of nanoparticulated glutathione for reducing oxidative stress in a body; and an effective amount of nanoparticulated 1-cysteine for reducing the oxidative stress in the body.

Example 49 is a composition as in any of Examples 41-48, wherein the nanoparticulated ingredient comprises a particle size that is sufficiently small such that the nanoparticulated ingredient passes through a lipid bilayer of a cell and enters an interior region of the cell.

Example 50 is a composition as in any of Examples 41-49, wherein the nanoparticulated ingredient comprises: the glutathione, wherein the glutathione comprises from about 0.5 vol % to about 5 vol % a total volume of the composition prior to addition of the water solvent; the l-cysteine, wherein the l-cysteine comprises from about 10 vol % to about 30 vol % the total volume of the composition prior to addition of the water solvent; and the curcumin, wherein the curcumin comprises from about 60 vol % to about 85 vol % prior to addition of the water solvent.

Example 51 is a composition as in any of Examples 41-50, further comprising calcium ascorbate, wherein the calcium ascorbate comprises from about 2 vol % to about 15 vol % a total volume of the composition prior to addition of the water solvent.

Example 52 is a composition as in any of Examples 41-51, wherein the nanoparticulated ingredient comprises the glutathione, and wherein the glutathione comprises from about 0.10 mol % to about 2.0 mol % the composition.

Example 53 is a composition as in any of Examples 41-52, wherein the nanoparticulated ingredient comprises the l-cysteine, and wherein the l-cysteine comprises from about 0.07 mol % to about 0.2 mol % the composition.

Example 54 is a composition as in any of Examples 41-53, wherein the nanoparticulated ingredient comprises the curcumin, and wherein the curcumin comprises from about 0.01 mol % to about 0.5 mol % the composition.

Example 55 is a composition as in any of Examples 41-54, wherein the composition comprises from about 92 mol % to about 99.8 mol % the water solvent.

Example 56 is a composition as in any of Examples 41-55, wherein the nanoparticulated ingredient comprises the glutathione, the l-cysteine, the curcumin, arginine, valine, proline, and glycine.

Example 57 is a composition as in any of Examples 41-56, further comprising collagen.

Example 58 is a composition as in any of Examples 41-57, further comprising hyaluronic acid.

Example 59 is a composition as in any of Examples 41-58, wherein the nanoparticulated ingredient comprises each of the glutathione and the l-cysteine; and wherein a volumetric ratio of the glutathione to the l-cysteine is from about 1:3 to about 3:1.

Example 60 is a composition as in any of Examples 41-59, wherein the nanoparticulated ingredient undergoes a nanoparticulation process comprising high-speed, high-sheer homogenization of a raw ingredient; and wherein the raw ingredient comprises one or more of calcium ascorbate, reduced glutathione (GSH), oxidized glutathione (GSSG), 1-glutathione, 1-cysteine, curcumin, arginine, valine, proline, glycine, hyaluronic acid, methylsulfonylmethane, collagen, or hydroxyproline.

Example 61 is a composition as in any of Examples 41-60, wherein the composition includes a formulation like any of the formulations described in any of Tables 1-17.

Example 62 is a composition as in any of Examples 41-60, wherein the composition is prepared according to any of the method steps described in any of Examples 21-40.

Example 63 is a method. The method includes providing an effective amount of a composition to an animal to reduce oxidative stress in the animal, wherein the composition comprises: a nanoparticulated ingredient, wherein the nanoparticulated ingredient comprises one or more of nanoparticulated glutathione, nanoparticulated l-cysteine, nanoparticulated curcumin, or nanoparticulated collagen; and a water solvent.

Example 64 is a method as in Example 63, wherein the composition includes any of the compositions described in any combination of any of Examples 41-60.

Example 65 is a method as in any of Examples 63-64, wherein the composition includes a formulation like any of the formulations described in any of Tables 1-17.

Example 66 is a method as in any of Examples 63-65, wherein the composition is prepared according to any of the method steps described in any of Examples 21-40.

Example 67 is a method as in any of Examples 63-66, wherein providing the effective amount of the composition to the animal comprises providing a daily dose to the animal.

Example 68 is a method as in any of Examples 63-67, wherein the animal is a horse, and wherein providing the effective amount of the composition to the animal comprises providing a daily dose to the horse by way of one or more of intramuscular administration, intravenous administration, or oral consumption.

Example 69 is a method as in any of Examples 63-68, wherein providing the effective amount of the composition to the animal comprises optimizing an amount of the composition provided to the animal based on current activity levels of the animal.

Example 70 is a method as in any of Examples 63-69, wherein providing the effective amount of the composition to the animal comprises: providing a standard daily dose to the animal when the animal is outside a threshold time period prior to an event; and providing an increased amount of the composition to the animal when the animal is within the threshold time period prior to the event.

Example 71 is a method as in any of Examples 63-70, wherein the animal is a horse, and wherein the event comprises one or more of a race, equestrian show, showjumping competition, or a show.

Example 72 is a method as in any of Examples 63-71, wherein the animal comprises a non-human animal, and wherein the non-human animal comprises one of a Equidae, canine, feline, bovine, or junglefowl.

Example 73 is a method as in any of Examples 63-72, wherein the animal comprises a human.

Example 74 is a method as in any of Examples 63-73, wherein providing the effective amount of the composition to the animal comprises providing a liquid dose of the composition to the animal, and wherein the liquid dose comprises a volume from about 5 mL to about 30 mL the composition.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations might be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A method comprising:
providing an effective amount of a composition to an animal to reduce oxidative stress in the animal, wherein the composition comprises:
a nanoparticulated ingredient, wherein the nanoparticulated ingredient comprises one or more of nanoparticulated glutathione, nanoparticulated l-cysteine, nanoparticulated curcumin, or nanoparticulated collagen; and
a water solvent.

2. The method of claim 1, wherein providing the effective amount of the composition to the animal comprises providing a daily dose to the animal.

3. The method of claim 1, wherein the animal is a horse, and wherein providing the effective amount of the composition to the animal comprises providing a daily dose to the horse by way of one or more of intramuscular administration, intravenous administration, or oral consumption.

4. The method of claim 1, wherein providing the effective amount of the composition to the animal comprises optimizing an amount of the composition provided to the animal based on current activity levels of the animal.

5. The method of claim 1, wherein providing the effective amount of the composition to the animal comprises:
providing a standard daily dose to the animal when the animal is outside a threshold time period prior to an event; and
providing an increased amount of the composition to the animal when the animal is within the threshold time period prior to the event.

6. The method of claim 5, wherein the animal is a horse, and wherein the event comprises one or more of a race, equestrian show, showjumping competition, or a show.

7. The method of claim 1, wherein the animal comprises a non-human animal, and wherein the non-human animal comprises one of a Equidae, canine, feline, bovine, or junglefowl.

8. The method of claim 1, wherein the animal comprises a human.

9. The method of claim 1, wherein providing the effective amount of the composition to the animal comprises providing a liquid dose of the composition to the animal, and wherein the liquid dose comprises a volume from about 5 mL to about 30 mL the composition.

10. The method of claim 1, wherein the water solvent comprises ultrapure water prior to addition of the nanoparticulated ingredient, and wherein the ultrapure water comprises a resistance of at least 14 megaohms prior to the addition of the nanoparticulated ingredient.

11. The method of claim 1, wherein the nanoparticulated ingredient comprises a particle size from about 10 nanometers to about 100 nanometers.

12. The method of claim 1, wherein the nanoparticulated ingredient comprises at least the nanoparticulated glutathione, and wherein the nanoparticulated glutathione reduced glutathione.

13. The method of claim 1, wherein the nanoparticulated ingredient comprises each of the nanoparticulated glutathione, the nanoparticulated l-cysteine, the nanoparticulated curcumin, and the nanoparticulated collagen.

14. The method of claim 1, wherein the nanoparticulated ingredient of the composition comprises:
an effective amount of the nanoparticulated glutathione for reducing oxidative stress in the animal; and
an effective amount of the nanoparticulated l-cysteine for reducing the oxidative stress in the animal.

15. The method of claim 1, wherein the nanoparticulated ingredient comprises a particle size that is sufficiently small such that the nanoparticulated ingredient passes through a lipid bilayer of a cell of the animal to enter an interior region of the cell.

16. The method of claim 1, wherein the composition is such that the nanoparticulated ingredient comprises:
the nanoparticulated glutathione, wherein the nanoparticulated glutathione comprises from about 0.5 vol % to about 5 vol % a total volume of the composition prior to addition of the water solvent;
the nanoparticulated l-cysteine, wherein the nanoparticulated l-cysteine comprises from about 10 vol % to about 30 vol % the total volume of the composition prior to addition of the water solvent; and
the nanoparticulated curcumin, wherein the nanoparticulated curcumin comprises from about 60 vol % to about 85 vol % prior to addition of the water solvent.

17. The method of claim 1, wherein the nanoparticulated ingredient comprises:
the nanoparticulated glutathione, wherein the nanoparticulated glutathione comprises from about 10 vol % to about 70 vol % a total volume of the composition prior to addition of the water solvent;
the nanoparticulated l-cysteine, wherein the nanoparticulated l-cysteine comprises from about 20 vol % to about 70 vol % the total volume of the composition prior to addition of the water solvent; and
the nanoparticulated curcumin, wherein the nanoparticulated curcumin comprises from about 1 vol % to about 5 vol % prior to addition of the water solvent.

18. The method of claim 1, further comprising calcium ascorbate, wherein the calcium ascorbate comprises from about 2 vol % to about 15 vol % a total volume of the composition prior to addition of the water solvent.

19. The method of claim 1, wherein the composition comprises from about 92 mol % to about 99.8 mol % the water solvent.

20. The method of claim 1, wherein the nanoparticulated ingredient comprises each of the nanoparticulated glutathione and the nanoparticulated l-cysteine; and
wherein a volumetric ratio of the nanoparticulated glutathione to the nanoparticulated 1-cysteine is from about 1:3 to about 3:1.
